(19) United States
(12) Patent Application Publication
(10) Pub. No.: US 2016/0331449 A1
(43) Pub. Date: Nov. 17, 2016

(54) EXPANDABLE ABLATION CATHETER

(71) Applicant: Cook Medical Technologies LLC, Bloomington, IN (US)

(72) Inventor: Tyler Evans McLawhorn, Winston-Salem, NC (US)

(21) Appl. No.: 15/153,928

(22) Filed: May 13, 2016

Related U.S. Application Data
(60) Provisional application No. 62/162,063, filed on May 15, 2015.

(51) Int. Cl. A61B 18/14

(52) U.S. Cl. CPC A61B 18/1492; A61B 2018/00577

(57) ABSTRACT

An expandable ablation catheter includes an expandable section having a plurality of channels defining struts. The expandable section has at least one conductor on one of the struts configured to couple to an energy source. The expandable section is expandable through longitudinal compression to expand the strut having the conductor outward to contact an inner surface of a lumen.

### REFERENCE TO EARLIER FILED APPLICATION

This application claims the benefit under 35 U.S.C. §119(e) of U.S. Provisional Patent Application No. 62/162,063, filed May 15, 2015, which is incorporated, in its entirety, by this reference.

### FIELD

Embodiments of the present invention relate to medical devices and more particularly to devices and methods for ablating tissue in a body lumen.

### BACKGROUND

The controlled removal or destruction of tissue is termed ablation. Ablation is sometimes used in medical procedures to alter the function of tissue. For example, removal of tissue near a nerve may disrupt the function of the nerve. It has been shown that high blood pressure may be related to hyperactivity of the renal sympathetic nerve. Therefore it is possible to treat high blood pressure by disrupting the function of the renal sympathetic.

The renal sympathetic nerve is actually a nervous system surrounding the renal arteries. The internal surface of the renal artery may be ablated to disrupt the function of the renal sympathetic nerve. Because the renal sympathetic nerve surrounds the renal artery, a successful ablation may ablate the tissue in at least a 360-degree arc around the artery. However, a circular ablation at a single location may cause the artery to stricture or narrow, or cause other complications. To avoid this problem, a helical section of tissue is ablated, such that the tissue is ablated in at least a 360-degree arc, but not in a single circular location.

There are currently at least two difference ways in which a helical ablation pattern may be applied to the internal surface of the nerve. In the first, a flexible electrode is placed on an outer surface of a balloon and is sized to form a helix against the surface of the artery when the balloon is inflated. The balloon is delivered to the treatment site in an uninflated state and is then inflated at the treatment site. Electrical energy is then applied to the helical conductor and the tissue proximate the conductor is ablated. While this provides for a complete ablation using a single application, blood flow is typically blocked for the duration of the ablation procedure. Additionally, the balloon and the helical conductor must be sized to match the internal diameter of the artery to ensure adequate contact with the tissue.

In another system, a narrow electrode is positioned on the distal end of a deflecting catheter. At the treatment site an operator deflects the electrode laterally until it reaches the internal surface of the artery. Electrical energy is then applied to the electrode and tissue near the tip is ablated. The catheter is then advanced axially and rotated to position the electrode in a new position adjacent the first ablation point. Electrical energy is applied again ablating the tissue adjacent the first ablation point. The catheter is advanced and rotated once again and the process is repeated until a full ablation is performed. This procedure is advantageous in that blood flow is continuous during the procedure, but is limited by the length of time it takes to perform the operation.

It would be beneficial to have a system that combines the speed of the balloon based system while still allowing blood to flow through the vessel like the deflecting catheter system.

### SUMMARY

In one aspect, an expandable ablation catheter is disclosed that includes an elongated member having a proximal end and a distal end; an expanding section having an expanding section distal end and an expanding section proximal end, the expanding section having a plurality of channels disposed at a distal portion of the elongated member, the plurality of channels defining a plurality of struts extending from the expanding section distal end to the expanding section proximal end and passing through the wall from an outside surface of the elongated member to an inside surface of the elongated member, each of the plurality of channels extending longitudinally and laterally in a general spiral shape; a conductor disposed on an exterior surface of a strut from among the plurality of struts, the conductor configured to be coupled to an energy source; and a compression mechanism configured to compress the expanding section from a first configuration in which the expanding section distal end and the expanding section proximal end are space apart a first distance to a second configuration in which the expanding section distal end and the expanding section proximal end are spaced apart a second distance less than the first distance.

In some embodiments, the compression mechanism comprises a second elongated member disposed within the bore, the second elongated member attached to the expanding portion distal end and movable from a first position in which the expanding section is in the first configuration and a second position in which the expanding mechanism is in the second configuration. In some embodiments, the compression mechanism comprises the expanding section being self-expanding, wherein the expanding section is self-biased to second configuration.

In some embodiments, the expandable ablation catheter further includes a sheath disposed about the catheter, the sheath slidable from a first position in which an inner wall of the sheath constrains the struts of the expanding and a second position in which the inner wall of the sheath does not constrain the struts of the expanding section.

In some embodiments, the expanding section is made of nylon.

In some embodiments, the expandable ablation catheter further includes a conductor disposed on the outer surface of at least one strut. In some embodiments, the conductor is a conductive ink. In some embodiments, a second conductor is disposed on the outer of a strut other than the at least one strut. In some embodiments, the at least one strut and the at least one other strut are adjacent struts. In some embodiments, the first conductor and the second conductor are in electrical communication with one another. In some embodiments, the first conductor and the second conductor are not in electrical communication with one another. In some embodiments, at least one of the plurality of channels extends greater than 360 degrees about the catheter.

In another aspect, a method for making an expanding catheter is disclosed. The method includes obtaining a catheter having at least one lumen; identifying an expanding section distal end; identifying an expanding section proximal end; forming a plurality of channels in the catheter, the channels extending longitudinally and laterally in a general spiral shape and defining a plurality of struts between the plurality of channels; obtaining a longitudinal member; and attaching a distal end of the longitudinal member to a portion of the catheter distal to expanding section distal end.

In some embodiments, the longitudinal member is disposed within a lumen from among the at least one lumen. In some embodiments, a conductor is attached to at least one of the plurality of struts, the conductor running along the at least one of the plurality of struts; and an electrical connector adapted to be coupled to an energy source is attached to the conductor.

In another aspect another method for making an expanding catheter is disclosed. The method includes obtaining an elongated member; obtaining a tubular segment having a bore; forming a plurality of channels into the tubular segment to define a plurality of struts between the plurality of channels; and attaching a distal end of the elongated member to a proximal end of the tubular segment.

In some embodiments a conductor is attached to at least one of the plurality of struts, the conductor running along the at least one of the plurality of struts; and an electrical connector adapted to be coupled to an energy source is attached to the conductor.

In some embodiments, the tubular segment with the plurality of spiral channels is longitudinally compressed to expand the plurality of struts laterally; and the compressed tubular segment is heated to shape set the plurality of struts in an expanded configuration.

In some embodiments, a second longitudinal member having a lumen sized to receive the unexpanded tubular segment is obtained and positioned over the tubular segment to constrain the struts to an unexpanded configuration.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 illustrates a side view of an expandable section of an expandable ablation catheter in an unexpanded configuration.

FIG. 2 illustrates a side view of the expandable section of the expandable ablation catheter of FIG. 1 in an expanded configuration.

FIG. 3 illustrates a side view of an expandable ablation catheter in an unexpanded configuration.

FIG. 4 illustrates a side view of the expandable ablation catheter of FIG. 3 in an expanded configuration.

FIG. 5 illustrates a side view of an expandable ablation catheter in an unexpanded configuration.

FIG. 6 illustrates a side view of the expandable ablation catheter of FIG. 5 in an expanded configuration.

FIG. 7 illustrates a side view of an expandable section of an expandable ablation catheter having a conductor on a strut.

FIG. 8 illustrates a side view of an expandable section of an expandable ablation catheter having conductors on adjacent struts.

FIG. 9 illustrates a side view of an expandable section of an expandable ablation catheter having a strut removed and conductors on adjacent struts.

FIG. 10 illustrates a schematic of a proximal end of an expandable ablation catheter.

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present disclosure are set forth in the attached figures and in the Detailed Description as provided herein and as embodied by the claims. It should be understood, however, that this Detailed Description does not contain all of the aspects and embodiments of the one or more present disclosure, is not meant to be limiting or restrictive in any manner, and that the disclosed herein is/are and will be understood by those of ordinary skill in the art to encompass obvious improvements and modifications thereto.

Additional advantages disclosed embodiments will become readily apparent from the following discussion, particularly when taken together with the accompanying drawings.

In the following discussion, the terms “distal” and “proximal” will be used to describe the opposing axial ends of the inventive balloon catheter, as well as the axial ends of various component features. The term “distal” is used in its conventional sense to refer to the end of the apparatus (or component thereof) that is furthest from the operator during use of the apparatus. The term “proximal” is used in its conventional sense to refer to the end of the apparatus (or component thereof) that is closest to the operator during use. For example, a catheter may have a distal end and a proximal end, with the proximal end designating the end closest to the operator during an operation, such as a handle, and the distal end designating an opposite end of the catheter, such as treatment tip. Similarly, the term “distally” refers to a direction that is generally away from the operator along the apparatus during use and the term “proximally” refers to a direction that is generally toward the operator along the apparatus.

In the following discussion, the term “elongated member” will be used to describe an elongated structure having a distal and proximal end. Examples of elongated members include tubes, catheters, rods, and so forth. An elongated member may include at least one lumen running the length thereof.

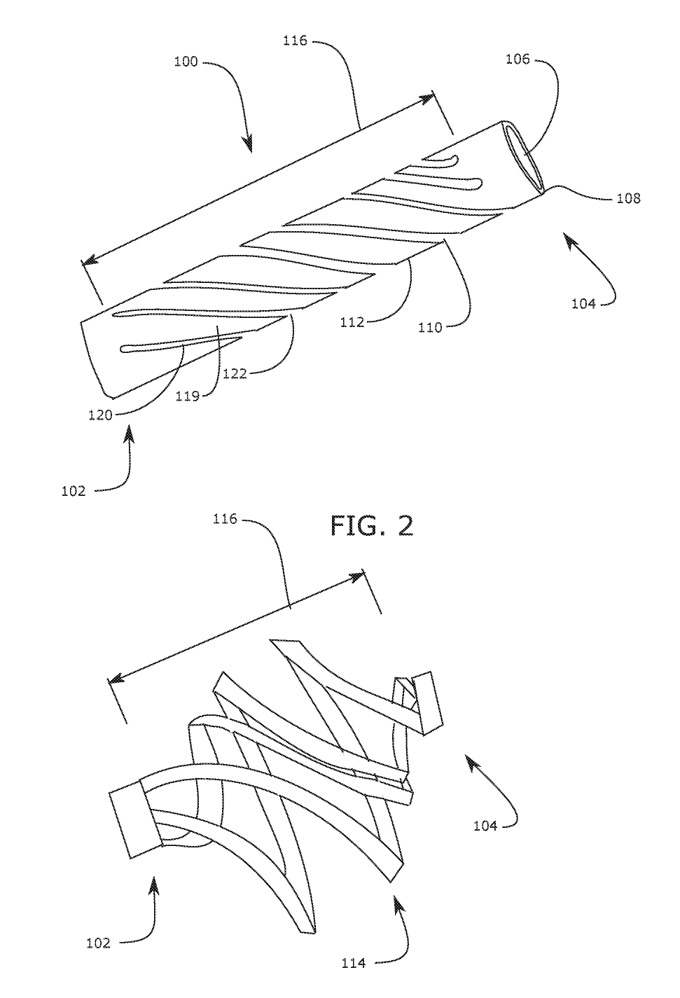

FIG. 1 illustrates an expandable section 100 of an expandable ablation catheter. The expandable section 100 has a proximal end 102 and a distal end 104. A bore 106 runs longitudinally within the expandable section 100 and is defined by a wall 108 of the expandable section 100. The expandable section 100 has a plurality of channels 110 extending from the proximal end 102 to the distal end 104 and that pass through the wall 108. The plurality of channels 110 may spiral around the outer surface of the expandable section 100 extending circumferentially and axially for an axial distance 116. In some embodiments, the plurality of channels 110 extend circumferentially a single turn around the outer surface, or in other embodiments the plurality of channels 100 extend more or less than a single turn. In still other embodiments, the plurality of channels 100 may have other shapes. The plurality of channels 110 define a plurality of struts 112 with a strut 119 being formed between adjacent channels 120, 122. Because the strut 119 is formed between the two adjacent channels 120, 122, the strut 119 follows the patters of the adjacent channels 120, 122, and in the embodiment of FIG. 1 the strut 119 also extends circumferentially and axially. The channels 120, 122 may have a uniform profile resulting in a uniform strut 119 profile, or in other embodiments the channels 120, 122 may have a varying profile resulting in a strut 119 that varies in profile. For example, the width of a strut 119 may be varied by varying the width of a channel 120, 122. The varying width of a strut 119 may allow the stiffness of a strut 119 to vary along the length of the expandable section 100.

FIG. 2 illustrates the expandable section 100 with the proximal end 102 and the distal end 104 having been moved towards one another to decrease the axial distance 116 of the plurality of channels 110. The length of each strut among the plurality struts 112 does not change with the compression of the axial distance 116 and each strut among the plurality of struts 112 extends radially proximate their midsection 114 to maintain the same length. In some embodiments, each of the struts is self-biased to the shape shown in FIG. 1, and an axially compressive force is required to move the distal end 104 towards the proximal end 102 resulting in the expanded shape shown in FIG. 2.

In other embodiments, each of the struts is self-biased to the configuration shown in FIG. 2. In these embodiments, the plurality of struts 112 requires an external biasing constraint to maintain the configuration shown in FIG. 1. When the external biasing constraint is removed, the plurality of struts return to the shape shown in FIG. 2.

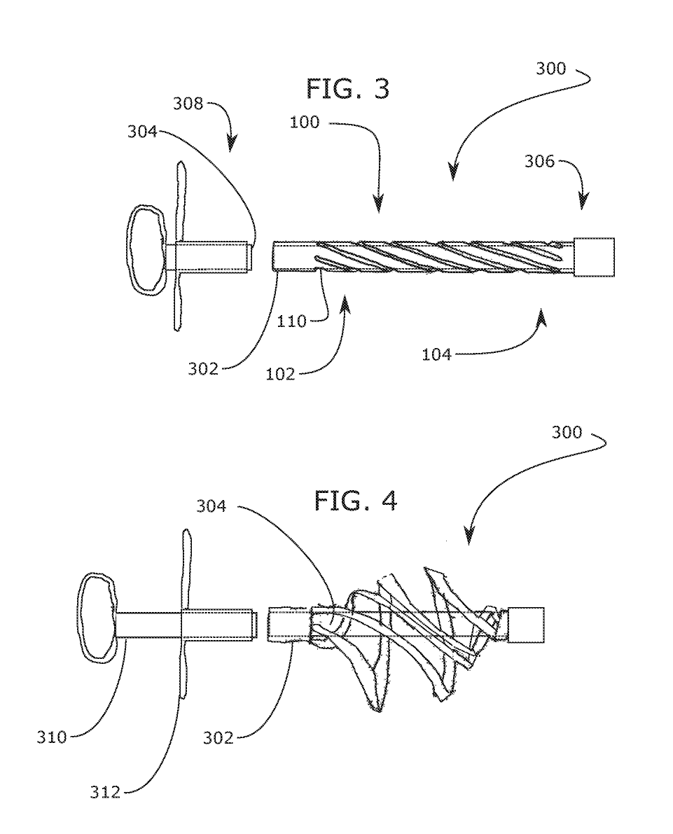

FIG. 3 illustrates an expandable ablation catheter 300 with an expandable section 100 as shown in FIG. 1 and FIG. 2. In this embodiment, the expandable section 100 is a portion of a first elongated member 302 in the form of a catheter. The expandable section 100 is formed in the first elongated member 302 by cutting spiral slots 110 in the expandable section 100. In other embodiments, the expandable section 100 may be formed separately and attached to the first elongated member 302 using conventional techniques such as adhesives, brazing, and crimping.

The first elongated member 302 has a lumen disposed longitudinally within it. A second elongated member 304 is disposed within the lumen and extends through the expandable section 100. The second elongated member 304 is free to move axially relative to a proximal portion 308 of the first elongated member 302. A distal end 306 of the second elongated member 304 is configured to affect longitudinal movement of the distal end 104 of the expandable section 100.

In one embodiment, the distal end 306 of the second elongated member 304 is adhered to the distal end 104 of the expandable section 100, such that a proximal movement of the second elongated member 304 results in the distal end 104 of the expandable section 100 moving toward the proximal end 102 of the expandable section 100. The second elongated member 304 may be adhered to the expandable section by conventional techniques such as adhesive, hot melt, and crimped connectors.

In another embodiment, the distal end 306 of the second elongated member 304 is expanded to have a diameter greater than an inner diameter of the distal end 104 of the expandable section 100. Axial movement in the distal direction will result in the second elongated member 302 extending beyond the distal end 104 of the expandable section 100, while axial movement in the proximal direction will result in the distal end 306 of the second elongated member 302 interfering with the distal end 104 of the expandable section 100, and thereby force the distal end 104 of the expandable section 100 to move proximally.

In the embodiment of FIG. 3 and FIG. 4, the expandable section 100 naturally maintains a first, unexpanded configuration shown in FIG. 1 and FIG. 3. When the expandable section 100 is axially compressed by proximal movement of the second elongated member 302 as shown in FIG. 4, the expandable section 100 is biased to a second, expanded configuration. The expandable section 100 is compressed by retracting a handle 310 relative to a grip 312. The handle 310 is coupled to the second longitudinal member 304 and the grip 312 is coupled to the first longitudinal member 302. When the handle 3310 is retracted it forces the distal end 306 of the second longitudinal member 304 to move proximally, compressing the expandable section 100 longitudinally. While the embodiment of FIG. 3 and FIG. 4 are shown using a handle 310 and a grip 312, embodiments are not so limited. For example, the second longitudinal member 304 may be coupled to any device capable of moving the second longitudinal member 304 proximally relative to the first longitudinal member 302. Or in some embodiments, it is possible that no device be coupled to the second longitudinal member 304 such that it is retracted directly.

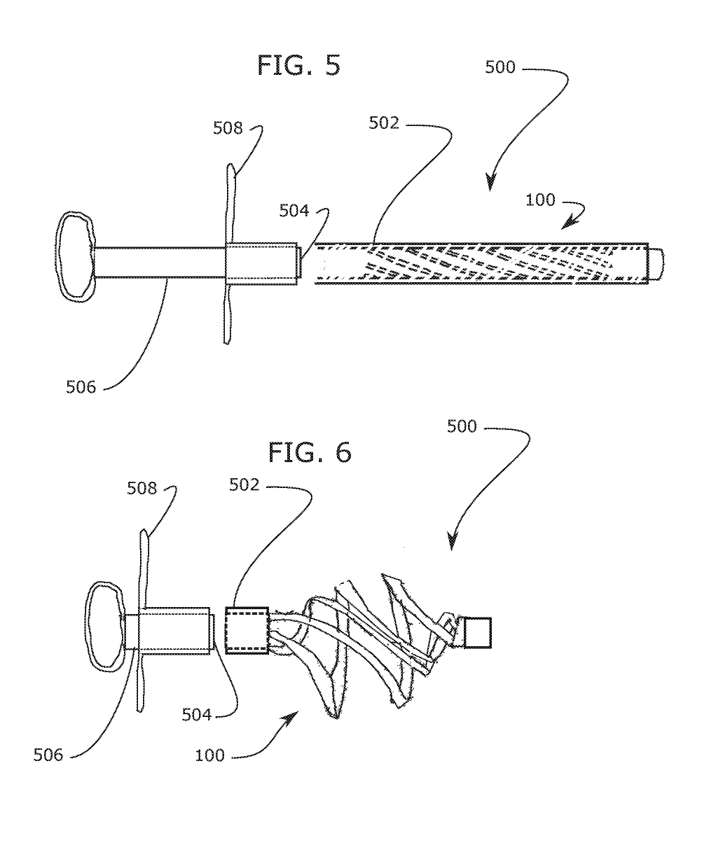

FIG. 5 and FIG. 6 illustrate another embodiment of an expandable ablation catheter 500. In this embodiment, the expandable section 100 is self-biased to the configuration shown in FIG. 6. In use, the expandable section 100 is compacted to the configuration shown in FIG. 5 for delivery to a treatment site. The expandable section 100 is kept compacted through a radial constraint provided by a sleeve 502. When the radial constraint is removed, such as by retracting the sleeve 502, the expandable section 100 expands to its relaxed, self-biased state shown in FIG. 6.

Expandable ablation catheter 500 includes a first longitudinal member in the form of a catheter 504 and a second longitudinal member in the form of sleeve 502. The catheter 504 is slidably disposed within the sleeve 502. The expandable section 100 of the catheter 504 is formed from a shape memory material such as a nickel titanium alloy. The remainder of the catheter 504 may be formed of the same material, or it may be a different material coupled to the expandable section 100. For example, the remainder of the catheter 504 may be nylon attached to the expandable section 100 through an adhesive.

Similar to the embodiment shown in FIG. 3 and FIG. 4, the embodiment of FIG. 5 and FIG. 6 includes a handle 506 and a grip 508. However, in this embodiment, the handle 506 is coupled to the catheter 504 and the grip 508 is coupled to the sleeve 502. The expandable section 100 of the expandable ablation catheter 500 is delivered to a treatment site in the configuration shown in FIG. 5, with the handle 506 protruding from the grip 508. Once at the treatment site, the grip 508 is retracted relative to the handle 506, moving the sleeve 502 proximally from the expandable section 100. Without the radial constraint provided by the sleeve 502, the expandable section 100 expands into its relaxed state shown in FIG. 6.

The disclosed embodiments are useful for delivering a helical ablation element to an inner surface of a vessel. Longitudinal compression of the expandable section 100 results in the struts expanding outward. The struts are selected so as to allow them to conform to the interior surface of a lumen. Thus, when the expandable section 100 is longitudinally compressed within a body lumen, the result is the forming a cylindrical surface in contact with an inner surface of the body lumen.

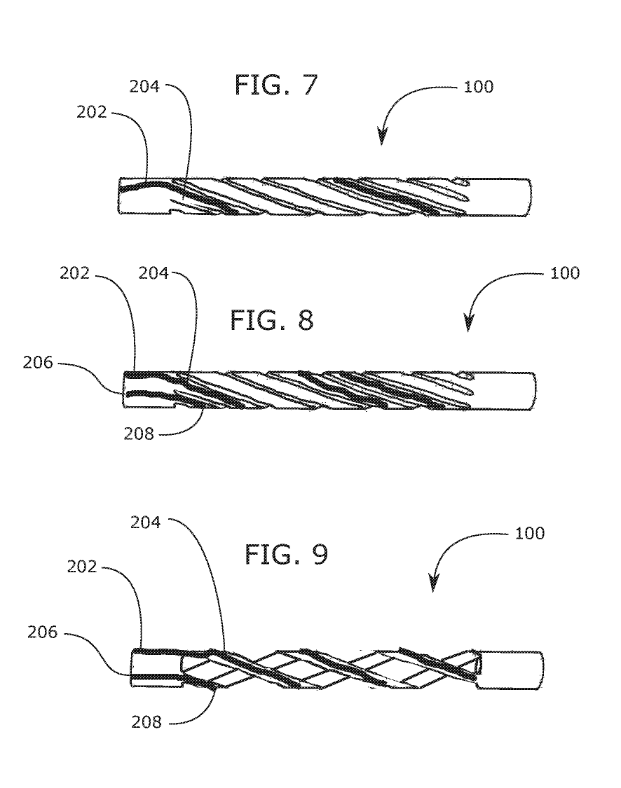

FIG. 7 to FIG. 9 illustrate three embodiments of an expandable section 100 having an ablation element in the form of a conductive coating 202 on a first strut 204. The conductive coating 202 may include a flexible base material adhered to the expandable section 100. One example of a suitable base material between the conductive coating 202 and the first strut 204 is silicone. In other embodiments, the ablation element is a separate conductive filament adhered to the first strut 204.

The conductive coating 202 may be a conductive ink applied to the strut first 204. One example of a conductive ink is silver ink, although other metallic inks are possible. The conductive coating 202 may comprise a conductive painting, conductive glue, or other conductive materials that form a conductive coating on the first strut 204.

FIG. 7 illustrates an exemplary embodiment of an expandable section 100 having a single pole ablation element on the first strut 204. The ablation element, be it a filament, conductive coating, or other element, is in electrical communication with an ablation power source located at a proximal end of the expandable ablation catheter. A second pole is located external to the patient and is placed in electrical communication with the patient's skin. The expandable section 100 of the expandable ablation catheter is delivered to a treatment area and energy is supplied to the ablation element. The energy passes from the ablation element through the patient's tissue and returns through the second pole.

FIG. 8 illustrates an exemplary embodiment of an expandable section 100 having a bipolar arrangement of ablation elements in the form of the conductive coating 202 on the first strut 204, and a second conductive coating 206 on a second strut 208. In this embodiment, two ablation elements are disposed on adjacent struts and are kept electrically insulated from one another. The ablation elements are each in electrical communication with a pole of the ablation power source. An ablation zone is formed between the first conductive coating 202 and the second conductive coating 206. This bipolar arrangement allows for a precise ablation zone between the conductive coatings 202, 206.

FIG. 9 illustrates an exemplary embodiment of an expandable section 100 having a bipolar arrangement of ablation elements in the form of the conductive coating 202 on the first strut 204, and a second conductive coating 206 on a second strut 208. In this embodiment, a third strut originally disposed between the first strut and the second strut has been removed, resulting in increased spacing between the first strut and the second strut. This results in a larger ablation zone.

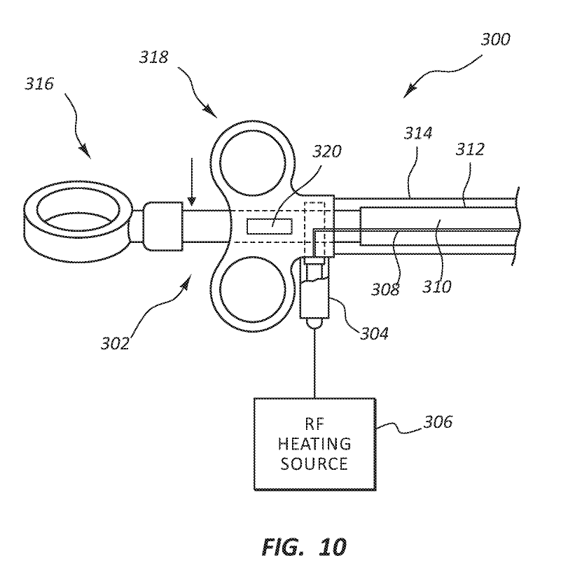

FIG. 10 illustrates an embodiment of a proximal end of an expandable ablation catheter. In each of the previously described embodiments, the ablation element is operably connected to an energy source. As shown in FIG. 10, a handle 302 may include a connector 304 for operably connecting the ablation element to an energy source 306. As shown, the energy source 306 may be a radio frequency source. However, other types of energy sources may also be used to provide energy to the ablation element. By way of non-limiting example, additional possible energy sources may include microwave and electric current. The ablation element is connected to the power source by an electrical conductor, such as one or more wires 308 that extend from the ablation element to the connector 304 that connects to the energy source 306. The one or more wires 308 may extend through a lumen 310 of an inner elongated member 312 or may extend through a lumen of an outer elongated member 314 or external to the outer elongated member 314 and may optionally include a sleeve surrounding the outer elongated member 314 and one or more wires 308.

As discussed above, the handle 302 is operable to move the inner elongated member 312 relative to the outer elongated member 314 so that the expandable section 100 moves between the expanded configuration and the collapsed configuration (see FIGS. 1 and 2). By way of non-limiting example, the handle 302 includes a first portion 316 and a second portion 318 that move relative to each other. As shown in FIG. 10, the first portion 316 is operably connected to the inner elongated member 312. The second portion 318 is operably connected to the outer elongated member 314. The first portion 316 may be moved proximally and/or the second portion 318 may be moved distally to move the inner elongated member 312 proximally and/or the outer elongated member 314 distally to move the expandable section 100 to the expanded configuration as shown in FIG. 2. As shown in FIG. 1, the first portion 316 may be moved distally and/or the second portion 318 moved proximally to move the inner shaft 312 distally and/or the outer shaft 314 proximally to move the expandable section 100 to the collapsed configuration.

The handle 302 may include a lock 320 shown to releasably lock the first portion 316 in position relative to the second portion 318 and thus lock the expandable section in position. The lock 320 may releasably lock the first and second portions 316, 320 of the handle 302 together at any proximal/distal positioning of the inner and outer elongated member 312, 314 so that the expandable section 100 may be locked at any size that is suitable for the treatment site. For example, if the treatment site is in a narrow lumen, the first portion 316 of the handle 302 may be moved slightly in the proximal direction to give the expandable section 302 a smaller diameter than if the first portion 316 were moved fully distally to give expandable section 100 the largest diameter.

Embodiments of the disclosure are further directed to a method for making an expandable ablation catheter. In the method a catheter is first obtained having at least one lumen. An expanding section distal end is identified along with an expanding section proximal end. A plurality of channels is then formed in the expanding section of the catheter. The channels may be helical in shape and spiral around the catheter for the length of the expanding section. A longitudinal member is then obtained and a distal end of the longitudinal member is attached to a portion of the catheter distal to expanding section distal end to form the expandable ablation catheter.

The longitudinal member may be narrower than the catheter and fit within the at least one lumen of the catheter. The longitudinal member would then extend the length of the lumen and through the expanding section.

The expandable ablation catheter may then have at least one conductor attached to at least one of the plurality of struts. The at least one conductor may then be coupled to an energy source to for providing ablative energy to the conductor.

In another method of making an expanding catheter, an elongated member is obtained along with a tubular segment having a bore. A plurality of channels is formed in the tubular segment resulting in a plurality of struts between the plurality of channels. A distal end of the elongated member is then coupled to a proximal end of the tubular segment.

A conductor may then be attached to at least one of the plurality of struts, with the conductor running along the at least one of the plurality of struts. An electrical connector is then coupled to the conductor with the electrical connector adapted to be coupled to an energy source to the conductor.

The tubular segment may be shape set to self-bias the plurality of struts expand laterally. To do so the tubular segment may be compressed longitudinally to expand the plurality of struts laterally and heated to set the plurality of struts in an expanded configuration. A second longitudinal member having a lumen sized to receive the unexpanded tubular segment is then positioned over tubular segment to constrain the struts to an unexpanded configuration.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

What is claimed:

1. A catheter comprising:
an elongated member having a proximal end and a distal end;
an expanding section having an expanding section distal end and an expanding section proximal end, the expanding section having a plurality of channels disposed at a distal portion of the elongated member, the plurality of channels defining a plurality of struts extending from the expanding section distal end to the expanding section proximal end and passing through the wall from an outside surface of the elongated member to an inside surface of the elongated member;
a conductor disposed on an exterior surface of a strut from among the plurality of struts, the conductor configured to be coupled to an energy source; and
a compression mechanism configured to axially compress the expanding section from a first configuration in which the expanding section distal end and the expanding section proximal end are space apart a first distance to a second configuration in which the expanding section distal end and the expanding section proximal end are spaced apart a second distance less than the first distance.

2. The catheter of claim 1, wherein the plurality of channels extend longitudinally and circumferentially in a spiral pattern.

3. The catheter of claim 1, wherein the compression mechanism comprises a second elongated member disposed within the bore, the second elongated member attached to the expanding portion distal end and movable from a first position in which the expanding section is in the first configuration and a second position in which the expanding mechanism is in the second configuration.

4. The catheter of claim 1, wherein the compression mechanism comprises the expanding section being self-expanding, wherein the expanding section is self-biased to second configuration.

5. The catheter of claim 4, further comprising a sheath disposed about the catheter, the sheath slidable from a first position in which an inner wall of the sheath constrains the struts of the expanding and a second position in which the inner wall of the sheath does not constrain the struts of the expanding section.

6. The catheter of claim 1, wherein the expanding section comprises nylon.

7. The catheter of claim 1, further comprising a conductor disposed on the outer surface of at least one strut.

8. The catheter of claim 7, wherein the conductor comprises a conductive ink.

9. The catheter of claim 7, further comprising a second conductor disposed on the outer of a strut other than the at least one strut.

10. The catheter of claim 9, wherein the at least one strut and the at least one other strut are adjacent struts.

11. The catheter of claim 9, wherein the first conductor and the second conductor are in electrical communication with one another.

12. The catheter of claim 9, where the first conductor and the second conductor are not in electrical communication with one another.

13. The catheter of claim 1, wherein at least one of the plurality of channels extends greater than 360 degrees about the catheter.

14. A method for making an expanding catheter, the method comprising:
obtaining a catheter having at least one lumen;
identifying an expanding section distal end;
identifying an expanding section proximal end;
forming a plurality of channels in the catheter, the channels defining a plurality of struts between the plurality of channels;
obtaining a longitudinal member; and
attaching a distal end of the longitudinal member to a portion of the catheter distal to expanding section distal end.

15. The method of claim 14, wherein the longitudinal member is disposed within a lumen from among the at least one lumen.

16. The method claim 14, further comprising:
attaching a conductor to at least one of the plurality of struts, the conductor running along the at least one of the plurality of struts; and
attaching an electrical connector adapted to be coupled to an energy source to the conductor.

17. A method of making an expanding catheter, the method comprising:
obtaining an elongated member;
obtaining a tubular segment having a bore;
forming a plurality of channels into the tubular segment to define a plurality of struts between the plurality of channels; and
attaching a distal end of the elongated member to a proximal end of the tubular segment.

18. The method claim 17, further comprising:
attaching a conductor to at least one of the plurality of struts, the conductor running along the at least one of the plurality of struts; and
attaching an electrical connector adapted to be coupled to an energy source to the conductor.

19. The method of claim 18, further comprising:
longitudinally compressing the tubular segment with the plurality of spiral channels to expand the plurality of struts laterally; and
heating the compressed tubular segment to shape set the plurality of struts in an expanded configuration.

20. The method of claim 19, further comprising:
obtaining a second longitudinal member having a lumen sized to receive the unexpanded tubular segment;
positioning the second longitudinal member over tubular segment thereby constraining the struts to an unexpanded configuration.
